(19) United States
(12) Patent Application Publication
(10) Pub. No.: US 2026/0108766 A1
(43) Pub. Date: Apr. 23, 2026

(54) ULTRASONIC TRANSDUCERS AND WIDE-AREA TRANSCRANIAL SONICATION DEVICE INCLUDING THE SAME

(71) Applicant: Seheun Choo, Seoul (KR)

(72) Inventor: Seheun Choo, Seoul (KR)

(21) Appl. No.: 19/356,264

(22) Filed: Oct. 13, 2025

(30) Foreign Application Priority Data
Oct. 17, 2024 (KR) 10-2024-0142206

(51) Int. Cl. A61N 7/00; B06B 1/06

(52) U.S. Cl. CPC A61N 7/00; B06B 1/0651; A61N 2007/0026

(57) ABSTRACT

Disclosed are a dry-coupling ultrasonic transducer that can deliver either focused and non-focused ultrasound to the brain without the use of hydrogel or degassed water as coupling media, while minimizing acoustic distortion, and a wide-area transcranial ultrasound sonication device that incorporates the ultrasonic transducer. The ultrasonic transducer includes a main body configured to generate ultrasound and a dry acoustic coupler configured to transmit the ultrasound to a sonication target, wherein the coupler includes a plurality of flexible coupling pins extending from the front surface of the transducer main body, to provide direct contact with the sonication target.

### CROSS-REFERENCE TO RELATED APPLICATIONS

This application claims the priority of Korean Patent Application No. 10-2024-0142206 filed on Oct. 17, 2024, in the Korean Intellectual Property Office, the disclosure of which is incorporated herein by reference.

### BACKGROUND

### Field

The present invention relates to an ultrasonic transducer and a wide-area transcranial sonication device incorporating the ultrasonic transducer, and more specifically, to an ultrasonic transducer capable of delivering ultrasound to the brain without using an aqueous coupling medium such as hydrogel, while minimizing acoustic field distortion, by directly contacting the surface of the sonication target, and to a wide-area transcranial sonication device incorporating the ultrasonic transducer.

### Description of the Related Art

Ultrasound is used for diagnostic and therapeutic applications or for non-destructive testing (NDT) that can inspect cracks/integrities of materials without disassembling them. Ultrasound is generated by a piezoelectric element in a transducer, and the generated ultrasonic pressure wave is reflected, refracted, and absorbed by differences in acoustic impedance (hereinafter referred to as impedance) of the transmitting medium. Reflection, refraction, and absorption of ultrasound are defined as distortion, and such distortion significantly reduces the efficiency and accuracy of ultrasound delivery.

Conventional ultrasound transducers use coupling materials with identical or similar impedance to the transmitting medium at the interface to minimize distortion during ultrasound transmission. Meanwhile, since the impedance of skin of the human body (15.3 Mega Ry) is similar to the impedance of water (14.8 Mega Ry), hydrogel containing water is most commonly used as an acoustic coupler for ultrasound delivery, for example, plastic bags containing degassed water or medical ultrasound gels (having a degree of fluidic viscosity).

Conventional acoustic coupling with the scalp for transcranial ultrasound delivery has used hydrogel or degassed water. However, microscopic air bubbles trapped between hairs on the scalp can significantly distort ultrasound transmission even in small amounts. This is due to a significant mismatch between the impedance of air at room temperature 20° C. (413 Ry) and the impedance of human tissue (15.3 Mega Ry), a difference of approximately 360,000 times or more.

For transcranial ultrasound delivery using conventional transducers, to facilitate ultrasound transmission, sonication must be applied through limited scalp areas without hair, through shaved areas, or along paths with minimal presence of hair. Additionally, when it is unavoidable to sonicate through areas with some hair, sufficient ultrasound gel must be applied to eliminate any air gaps along the ultrasound path.

This conventional transcranial sonication approach not only makes hair removal impractical, but also makes it difficult to deliver sufficient acoustic energy to the desired target area, restricting the sonicated area. Additionally, ultrasound gel, because of its fluidic properties, may flow between the scalp and the transducer immediately after application or dry out over time, requiring repeated application.

To address these problems, there is a need for a transducer that does not use hydrogel—that is, one that uses a dry acoustic coupling method—and a corresponding transcranial ultrasound sonication device incorporating such a transducer. Furthermore, such a device should be able to simultaneously/sequentially deliver ultrasound to various regions across the brain. There is also an urgent need for a dry-coupling device that directly contacts the scalp without using any hydrogel or degassed water. Additionally, such a device should be configured to minimize acoustic distortion and energy loss by conforming to the curvature of the head or skull, so that the coupler maintains direct contact with the scalp.

### RELATED ART DOCUMENT

### Patent Document

(Patent Document 1) Korean Patent Publication No. 10-2020-0063460 (published Jun. 5, 2020)
(Patent Document 2) Korean Patent Publication No. 10-2021-0007548 (published Jan. 20, 2021)

### SUMMARY

An object of the present invention for solving the above-mentioned problems is to provide a dry-coupling ultrasonic transducer that can deliver ultrasound pressure waves to the brain regions with minimal acoustic distortion, without using hydrogel or degassed water, by directly contacting the scalp or skin, and to provide a wide-area transcranial ultrasound sonication device incorporating the ultrasonic transducer.

Another object of the present invention is to provide a wide-area transcranial ultrasound sonication device capable of delivering ultrasound across extensive brain regions either simultaneously or sequentially.

Yet another object of the present invention is to provide a wide-area transcranial ultrasound sonication device that allows for adjustable angulation of an individual transducer to conform to the curvature of the skull.

To achieve the above objectives, an ultrasonic transducer according to an embodiment of the present invention includes a main body that generates ultrasound and a coupler configured to transmit the ultrasound generated by the main body to a brain region, wherein the coupler includes a plurality of coupling pins extending/protruding from the front surface of the main body to make direct contact with the sonicated object.

Each of the plurality of coupling pin is made of a flexible material that deforms upon contact with the sonicated object.

The tip of each of the plurality of coupling pins terminates in a curved surfaces with a predetermined radius of curvature.

The plurality of coupling pins are arranged with equal spatial spacing.

Each of the plurality of coupling pins is spaced at intervals between 1/10 and ½ of the ultrasound wavelength in the sonicated medium.

The main body of ultrasound transducer comprises a planar piezoelectric element, and the plurality of coupling pins have different lengths relative to one another, with the distal end portions collectively forming a concave shape to adjust the shape of ultrasound beam profile.

The main body comprises a non-planar piezoelectric element concave toward the sonicated target to focus ultrasound, and the distal ends of the coupling pins are arranged on a common surface.

To achieve the above-mentioned objectives, an ultrasonic transducer according to an embodiment of the present invention comprises a main body that generates ultrasound, a coupler module detachably mounted on the front surface of the main body, and a plurality of coupling pins protruding from the coupler module to contact the scalp or skin directly.

The configuration of the coupler module includes a first coupler module in which the contact surfaces of the plurality of coupling pins are positioned on the same plane as each other.

The configuration of the coupler module includes a second coupler module in which the distal end of the plurality of coupling pins form a concave shape.

The configuration of the coupler module further includes a third coupler module in which the distal end portions of the coupling pins form a concave shape, wherein the coupling pins protruding to different lengths from the coupling pins of the second coupler module to accommodate a different concave shape.

To achieve the above-mentioned objectives, a wide-area transcranial ultrasound sonication device according to an embodiment of the present invention includes headgear and an ultrasonic transducer mounted on the headgear and delivering ultrasound to biological tissue beneath the scalp, wherein the ultrasonic transducer includes a main body having a piezoelectric element that generate ultrasound based on electrical signal input and an acoustic coupler mounted on the front surface of the main body to transmit the ultrasound generated by the main body to the biological tissue, and the coupler incorporating a plurality of coupling pins that make direct contact with the scalp.

The ultrasonic transducer is mounted to the headgear via a swivel connector on the inside of the headgear, enabling angle adjustment to conform to the curvature of the skull.

A plurality of ultrasonic transducers are installed on the headgear.

The plurality of ultrasonic transducers are electrically connected to a single control device via a 1-to-n signal demultiplexer.

According to the ultrasonic transducer and the wide-area transcranial ultrasound sonication device incorporating the ultrasonic transducer of the present invention, ultrasound distortion can be prevented or minimized although hydrogel or degassed water are not used as an acoustic coupler.

According to the wide-area transcranial ultrasound sonication device of the present invention, ultrasound can be delivered across various regions in the brain, either simultaneously or sequentially.

According to the wide-area transcranial ultrasound sonication device of the present invention, the angle of each transducer can be adjusted to conform to the curved surface of the skull.

According to the wide-area transcranial ultrasound sonication device of the present invention, as the coupling pins of the coupler/coupler modules directly contact the scalp, distortion and energy loss of ultrasound propagation through the sonicated object can be minimized.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view showing an ultrasonic transducer according to the present invention.

FIG. 2 is a schematic diagram describing a conventional transducer main body.

FIG. 3 is a schematic diagram describing a transducer according to the present invention.

FIG. 4 is a view showing the arrangement of coupling pins according to the present invention.

FIG. 5 is a view showing an embodiment of a coupler according to the present invention.

FIG. 6 is a view showing another embodiment of a coupler according to the present invention.

FIG. 7 is a view showing an ultrasonic transducer according to another embodiment of the present invention.

FIG. 8 is a schematic diagram describing an ultrasonic transducer according to another embodiment of the present invention.

FIG. 9 is a view showing the first embodiment of a coupler module according to another embodiment of the present invention.

FIG. 10 is a view showing the second and third embodiments of a coupler module according to another embodiment of the present invention.

FIGS. 11 and 12 are views showing a wide-area transcranial ultrasound sonication device according to the present invention.

### DETAILED DESCRIPTION OF THE EMBODIMENT

Hereinafter, embodiments of the present invention will be described in detail through illustrative drawings. In adding reference numerals to components in each drawing, it should be noted that the same components are assigned with the same numerals as much as possible even if displayed in different drawings.

In describing embodiments of the present invention, when it is determined that a detailed description of related known configurations or functions would obscure understanding of the embodiments, such details, thereof, are omitted.

Furthermore, in describing the components of embodiments of the present invention, terms such as first, second, A, B, (a), (b), etc., may be used. These terms are only for distinguishing one component from other components, and the essence, order, or sequence of the corresponding component is not limited by the term.

In this specification, singular forms also include plural forms unless specifically stated otherwise in the phrase. As used in the specification, “comprises” and/or “comprising” does not exclude the presence or addition of one or more other components in addition to the mentioned components.

Hereinafter, the present invention will be described in more detail with reference to the attached drawings.

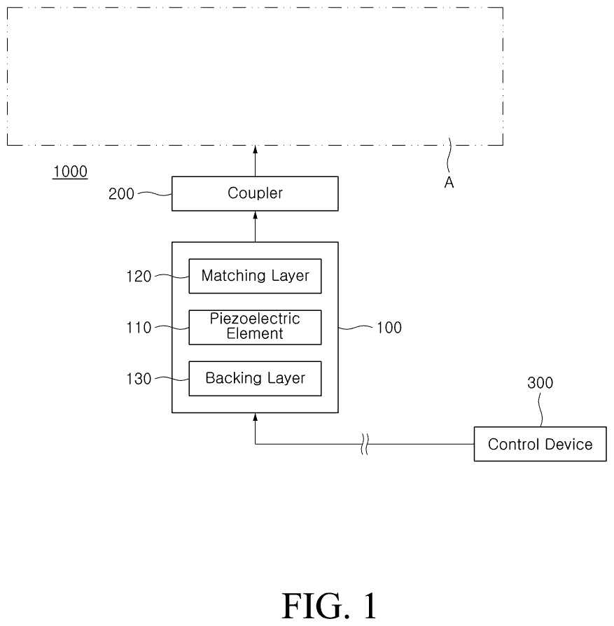

FIG. 1 is a view showing an ultrasonic sonication device 1000 (hereinafter also referred to as ‘transducer 1000’) according to the present invention.

Referring to FIG. 1, the ultrasonic sonication device 1000 according to the present invention includes a transducer main body 100 and a coupler 200 disposed on the front surface of the transducer main body 100. The ultrasonic transducer 1000 may further include a control device 300 that transmits electrical signals to the transducer main body 100.

The transducer main body 100 includes a piezoelectric element 110 that receives electrical signals from the control device 300 and vibrates to generate ultrasound. The piezoelectric element 110 converts electrical energy received from the control device 300 into mechanical energy. Electrical current is applied to the piezoelectric element 110 by the electrical signal received from the control device 300, and the piezoelectric element 110 repeatedly expands and contracts by the oscillating electrical currents, generating ultrasound. The piezoelectric element 110 operates using the piezoelectric effect, and as an example, PZT (Lead Zirconate Titanate) ceramic may be used, but is not limited thereto. The piezoelectric effect refers to a phenomenon in which current is generated when pressure is applied, and conversely, an object is deformed when current is applied. When current is applied to the piezoelectric element 110, the element vibrates. If this vibration frequency reaches ultrasound frequency (greater than 20,000 Hz), ultrasound is generated.

A backing layer 130 including a backing material is installed at the rear end of the piezoelectric element 110. The backing layer 130 is installed at the side of the piezoelectric element 110, opposite to the sonicated object, to absorb unnecessary vibrations of the piezoelectric element 110 and assist in forming an ultrasound beam. As an example, the backing material is composed of a material with high sound absorption such as a mixture of epoxy resin and metal particles, or filled with air, to prevent ultrasound generated by the vibration of the piezoelectric element 110 from being reflected to the rear surface of the transducer 1000.

A matching layer 120 that matches the impedance between the piezoelectric element 110 and the sonication target A is installed at the front end of the piezoelectric element 110. The matching layer 120 reduces the impedance difference between the piezoelectric element 110 and the sonication target A at the front end of the piezoelectric element 110 to increase the transmission efficiency of ultrasound energy. As an example, the matching layer 120 may be made of a polymer material and may be composed of multiple layers.

The transducer main body 100 may further include electrical terminals (not shown) connected by a cable that supports electrical signal transmission and reception to/from the control device 300, and a housing (not shown) that protects components such as the piezoelectric element 110 in the transducer main body 100 from the external environment.

The acoustic coupler 200 is installed on the front surface of the transducer main body 100. The coupler 200 transmits the ultrasound generated by the transducer main body 100 to the sonication target A. The sonication target A may be biological tissue, including the scalp and skin, but is not limited thereto and may include all materials that can be inspected in non-destructive testing (NDT).

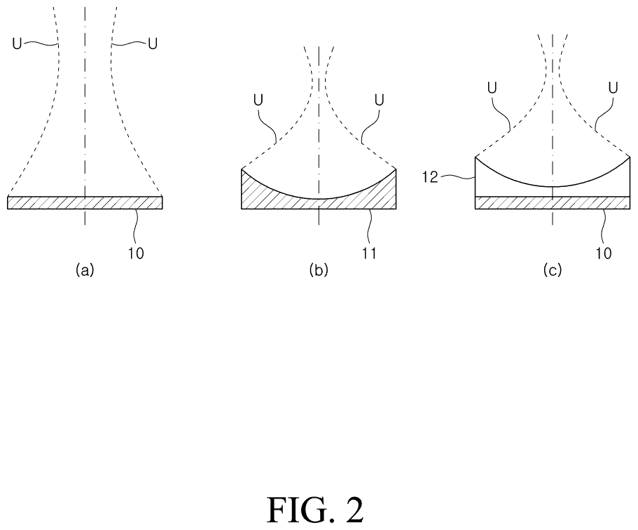

FIG. 2 is a schematic diagram briefly showing a conventional transducer main body. In FIG. 2, the transducer main body 100 of the transducer is briefly illustrated piezoelectric elements 10, 11 included in the transducer. The description of matching layers and backing layers are omitted from the illustration.

To pass ultrasound through the human skull, the operating frequency of the piezoelectric element is generally less than 900 kHz. The longer the wavelength of the sound wave, the less the absorption of ultrasound by the skull. The transducer includes piezoelectric elements 10, 11 that generate sound waves.

Referring to FIG. 2(a), (b), and (c), conventional transducers include planar piezoelectric elements 10 (refer to FIG. 2(a)) in which single or multiple piezoelectric elements are arranged in a planar or linear array, curved piezoelectric elements 11 (refer to FIG. 2(b)) in which piezoelectric elements are arranged in a curved array, and a configuration in which an acoustic lens 12 is attached in front of the planar piezoelectric element 10 (refer to FIG. 2(c)).

When the piezoelectric element is arranged in a plane, as shown in FIG. 2(a), the sound field distribution U of ultrasound generated from the piezoelectric element is loosely focused. For example, the intensity of ultrasound generated from the planar piezoelectric element in FIG. 2(a) becomes maximum at the end of the near field, then begins to decrease in the far field. The profile of the sound field U in the attached drawings is indicated by the dotted lines.

To deliver ultrasound generated by the piezoelectric element to a specific area, a focused ultrasound method is sometimes used, which focuses the sound field distribution U toward a specific focus by using a curved piezoelectric element 11 as shown in the transducer of FIG. 2(b), or by installing an acoustic lens 12 on the front surface of the planar piezoelectric element 10, that is, on the front surface of the piezoelectric element 10 opened toward the sonication target A, as shown in FIG. 2(c).

Ultrasound generated by the piezoelectric element is transmitted to the open front surface of the transducer main body 100. When ultrasound generated by transducers transmits energy using air as a medium, large impedance difference between air and body tissue leads to blocking most of the ultrasound energy without reaching the scalp or skin.

Therefore, acoustic coupling with the transducer main body 100 is essential to deliver ultrasound through the skin and skull. Conventional coupling method, using media such as hydrogel or degassed water, has been deployed to reduce the impedance difference between the transducer and body tissue.

That is, ultrasound is generated by applying electrical signals to the piezoelectric element, the generated ultrasound is transmitted into body tissue through the coupling medium with a small impedance difference. Within body, ultrasound may affect the biological tissue while undergoing various phenomena such as absorption, reflection, and refraction. For example, ultrasound reflected at the tissue boundaries returns to the transducer main body 100, and images can be generated based on detecting the returned ultrasound echoes.

The coupling 200 material should be carefully selected for efficient operation with the transducer main body 100. The main requirements are that the coupling method should minimize distortion/energy loss of ultrasound, should not adversely affect the directly contacted skin (causing pain or skin diseases), and should be deployable in extended duration of usage (for example, an hour at a time). These conditions should also apply to transcranial ultrasound technology; however, the hairs on the scalp pose important challenges.

That is, to facilitate ultrasound transmission, conventional approaches require that sonication path must be limited to cross areas without hair, require shaving of the site, or selecting path with minimal amount of hair. Additionally, when it is unavoidable to sonicate through areas with some hair, sufficient ultrasound gel must be applied to eliminate any air gaps along the acoustic path. These conventional methods not only make hair removal impractical, but also impede achieving a sufficient ultrasound field in the desired area. Furthermore, low-viscosity ultrasound gel may flow between the skin and the transducer immediately after application or dry out over time.

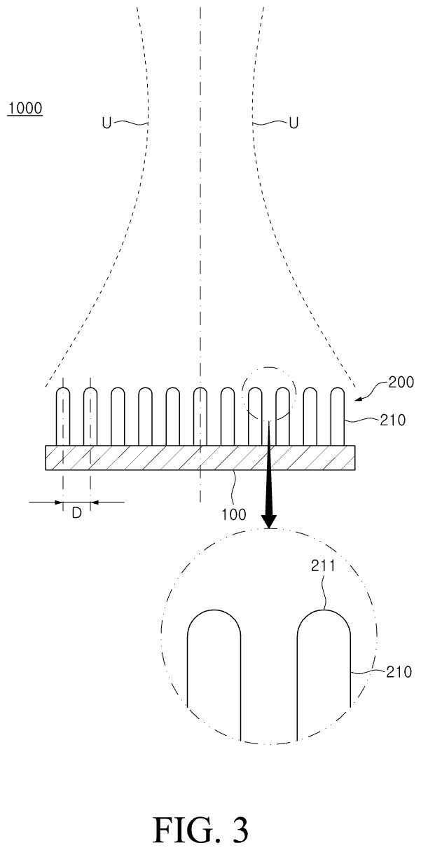

To solve the problems, the acoustic coupler 200 of the transducer 1000 according to the present invention includes a plurality of dry-coupling pins 210 (refer to FIG. 3) extending from the front surface of the transducer main body 100 toward the sonication target A to establish direct contact with the sonication target A.

FIG. 3 is a schematic diagram briefly showing a transducer 1000 according to the present invention.

Referring to FIG. 3, the acoustic coupler 200 according to the present invention includes a plurality of coupling pins 210 extending from the front surface of the transducer main body 100 toward the sonication target A for direct contact with the sonication target A. The plurality of coupling pins 210 vibrate together according to the operating frequency of the piezoelectric element 110. One distal end portion 211 of each of the plurality of coupling pins 210 contacts the scalp or skin and serves an acoustic point source that transmits the pressure wave originating from the piezoelectric element 110.

Each of the plurality of coupling pins 210 is made of a flexible material, and may be bent/compressed when contacting the sonication target A. One distal end portion 211 of each of the plurality of coupling pins 210 directly contacts the scalp surface between hairs or compresses the hair. One distal end portion 211 of each of the plurality of coupling pins 210 is lightly pressed against the scalp and can be elastically deformed reversibly upon pressing.

The plurality of coupling pins 210 may be made of soft plastic, silicone, or silicone with added doping material having acoustic properties similar to body tissue while allowing for predetermined elasticity and temporary deformation. Therefore, the plurality of coupling pins 210 can transmit ultrasound to body tissue by directly contacting the scalp while pressing hairs or the skin between hairs.

The distal tip 211 of each of the plurality of coupling pins 210 is mechanically coupled to the front surface of the transducer main body 100, and vibrate together according to the operating frequency of the piezoelectric element 110.

One distal tip 211 of each of the plurality of coupling pins 210 is formed with a curved surface having a predetermined curvature to prevent irritation to the scalp or skin. The distal tip 211 of each of the plurality of coupling pins 210 may have a diameter of approximately 1 to 2 mm as an example.

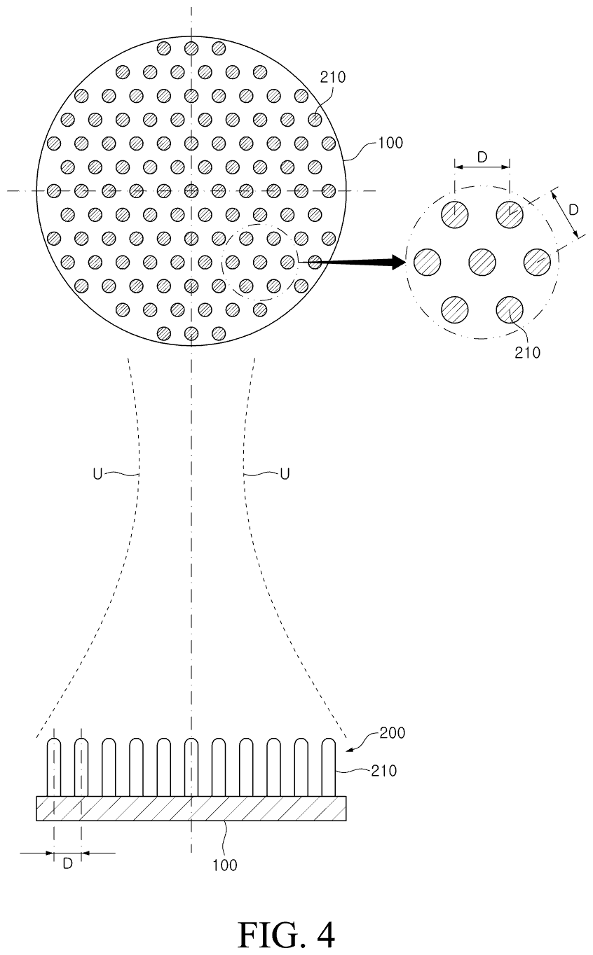

FIG. 4 is a view showing the arrangement of dry acoustic coupling pins 210 according to the present invention.

As shown in FIGS. 3 and 4, the coupler 200 according to the present invention includes a plurality of coupling pins 210 arranged at regular intervals.

The plurality of coupling pins 210 are arranged with equal spatial spacing on the front surface of the transducer main body 100. Specifically, the plurality of coupling pins 210 may be spaced at intervals between 1/10 and ½ of the ultrasound wavelength in the sonication target A.

As an example, when ultrasound wave is given at 400 kHz, the wavelength in the human body is about 3.75 mm (speed of sound wave in the human body 1500 m·s⁻¹/400000 s⁻¹). In this case, the spatial interval between the coupling pins 210 should be less than 1.87 mm. As another example, when the sound wave is 250 kHz, the wavelength in the human body is about 6 mm (speed of sound wave 1500 m·s⁻¹/250000 s⁻¹), so the interval should be less than 3 mm. If the spatial interval between the plurality of coupling pins 210 is larger than ½ of the wavelength in the sonicated medium, substantial distortion of sound waves occurs due to interference between sound waves and formation of side lobes within the sonicated object.

To maintain a constant spatial interval between the coupling pins 210, as an example, the coupling pins 210 may be arranged to have a hexagonal distribution based on triangular points es as basic units (refer to the enlarged view in FIG. 4).

The opposite side of the plurality of coupling pins 210 are manufactured to evenly and directly contact a plane or curved surface, conforming to the front surface of the transducer 1000, which is changed according to the type of piezoelectric element 110 in the transducer 1000, that is, the type of planar piezoelectric element (refer to 10 in FIG. 2(a)) and curved piezoelectric element (refer to 11 in FIG. 2(b)).

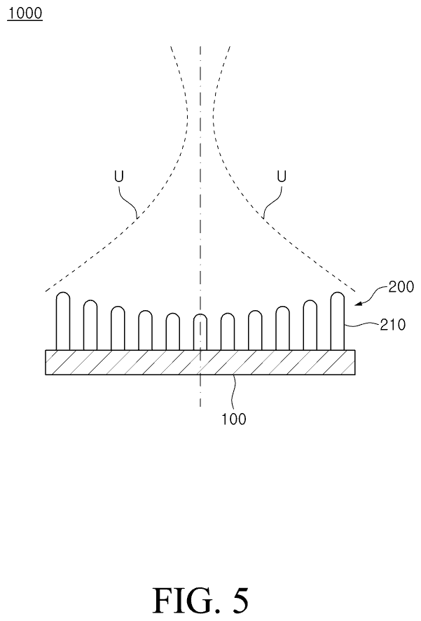

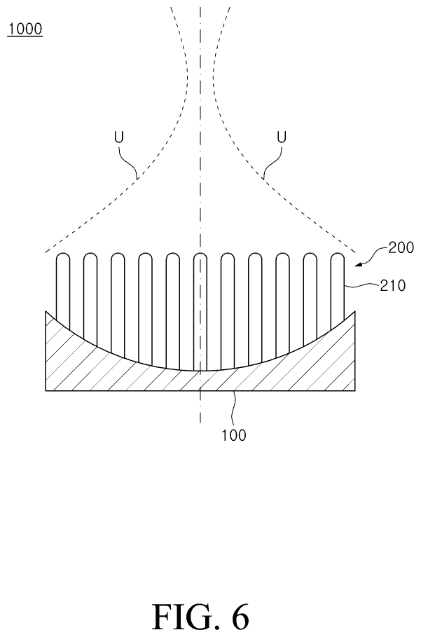

FIG. 5 is a view showing an embodiment of a dry acoustic coupler 200 according to the present invention, and FIG. 6 is a view showing another embodiment of the coupler 200 according to the present invention.

Referring to FIGS. 5 and 6, the surface distribution of distal tip 211 of the plurality of coupling pins 210, forming the front surface of the coupler 200 (which contacts the surface of the sonication target A) is distributed to form a plane or curved surface to conform to the scalp A or skin A. This configuration may focally deliver ultrasound to a specific region within body tissue.

FIG. 5 shows a transducer main body 100 including a planar piezoelectric element 110 and a plurality of coupling pins 210 installed on the front surface of the transducer main body 100. As described above, the piezoelectric element 110 can be a single planar element or linear array. When the transducer main body 100 includes a planar piezoelectric element 110, the plurality of coupling pins 210 are each configured with different protruding lengths to each other, and the end portions of the coupling pins 210 form a concave shape to deliver ultrasound toward a specific focal point. When the end portions of the plurality of coupling pins 210 form a concave shape, the focusing or dispersion direction of ultrasound propagation can be controlled by adjusting the protruding length of the plurality of coupling pins 210, which changes the curvature of the coupling surface.

FIG. 6 shows a transducer main body 100 including a curved piezoelectric element 110 and a plurality of coupling pins 210 installed on the front surface of the transducer main body 100. As described above, the curved piezoelectric element 110 may focally deliver acoustic waves in which multiple piezoelectric elements 110 are arranged in a curved array. When the transducer main body 100 includes a curved piezoelectric element 110, the distal ends 211 of the plurality of coupling pins 210 form a planar shape contacting the planar sonication target A. The curved piezoelectric element 110 has a concave shape toward the target A to focus the ultrasound onto a specific focal point within. The focusing or dispersion direction of ultrasound propagation can be controlled by adjusting the curvature of the piezoelectric element 110.

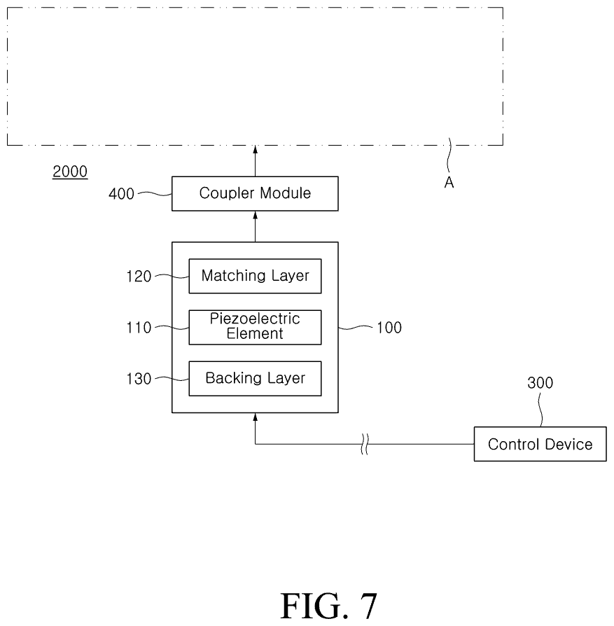

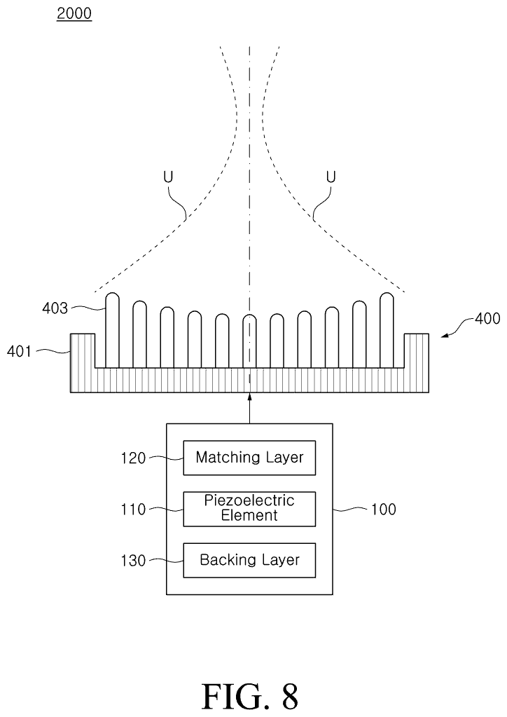

FIG. 7 is a view showing an ultrasonic sonication device 2000 (hereinafter also referred to as ‘transducer 2000’) according to another embodiment of the present invention, and FIG. 8 is a schematic diagram briefly showing an ultrasonic sonication device 2000 according to another embodiment of the present invention.

Referring to FIGS. 7 and 8, the ultrasonic sonication device 2000 according to another embodiment of the present invention includes a transducer main body 100 that generates ultrasound and a coupler module 400 detachably mounted to the front surface of the transducer main body 100. The ultrasonic sonication device 2000 according to another embodiment of the present invention may further include a control device 300 that transmits electrical signals to the transducer main body 100. Since the descriptions of the transducer main body 100 and the control device 300 are the same as described above, detailed descriptions thereof will be omitted.

The coupler module 400 includes a coupler module main body 401 and a plurality of coupling pins 403.

The coupler module main body 401 is detachably mounted to the front surface of the transducer main body 100. On the front surface of the coupler module main body 401, a plurality of coupling pins 403 protrudes from the coupler module main body 401 and directly contacts the scalp or skin.

The coupler module main body 401 may be detachably mounted to the front surface of the transducer main body 100 through known methods of mechanical attachment. For example, mechanical coupling may include mechanical fastening/interlock and/or bolted screws, and electrical coupling means may include electro-/permanent magnets. However, any coupling means that can detachably couple the transducer main body 100 and the coupler module main body 401 to each other is acceptable.

The plurality of coupling pins 403 vibrate together with the coupler module main body 401, where each pin serves as a ultrasound point source that transmits the pressure wave generated by the piezoelectric element 110 to the brain via direct contacts with the scalp or skin.

The plurality of coupling pins 403 protruding from the front surface of the coupler module main body 401 have the same characteristics as the plurality of coupling pins 403 described above. Therefore, detailed descriptions of the plurality of coupling pins 403 are omitted.

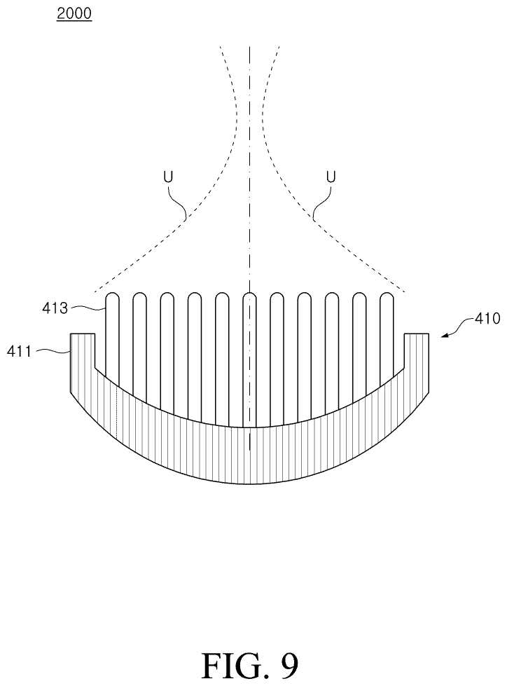

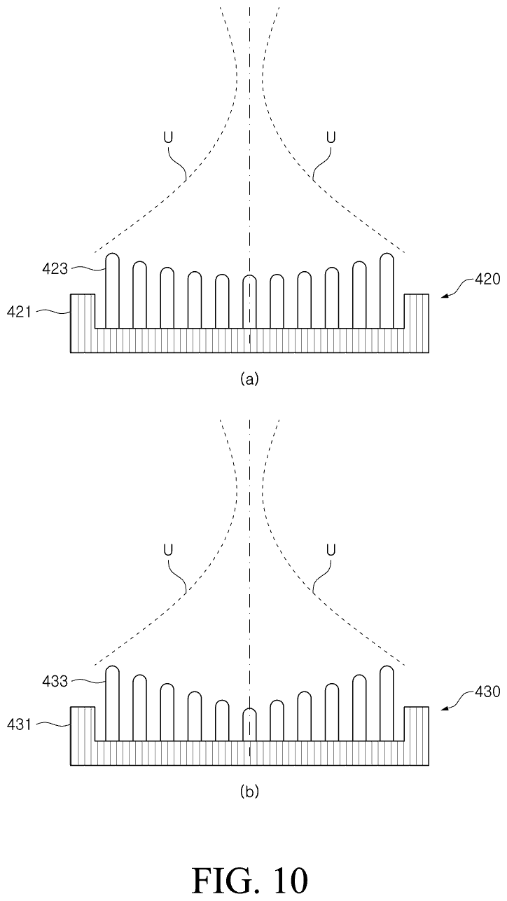

FIG. 9 is a view showing a first embodiment 410 of a coupler module 400 according to another embodiment of the present invention, and FIG. 10 is a view showing a second embodiment 420 and a third embodiment 430 of a coupler module 400 according to another embodiment of the present invention.

The coupler module 400 has various embodiments 410, 420, 430.

As a first embodiment of the coupler module 400 (first coupler module 410), as shown in FIG. 9, the first coupler module 410 includes a curved coupler module main body 411 and a plurality of coupling pins 413 protruding from the coupler module main body 411 toward the front surface, wherein the distal end of the plurality of coupling pins 413 are positioned on the same plane. In the coupling module 410 according to the first embodiment, the surface of the plurality of coupling pins 413 that contact the scalp A or skin A, as a whole, share the same plane. The coupler module 410 according to the first embodiment may be mounted on the front surface of the transducer main body 100 (a curved piezoelectric element). The curved coupler module main body 411 may have a shape to conform to the front surface of the transducer main body 100, having the curved piezoelectric element 110.

As a second embodiment of the coupler module 400 (second coupler module 420), as shown in FIG. 10(a), the second coupler module 420 includes a planar coupler module main body 421 and a plurality of coupling pins 423 extending from the coupler module main body 421 toward the front surface, wherein the distal end of the plurality of coupling pins 423 form a concave shape. The coupler module 420 according to the second embodiment has the plurality of coupling pins 423 each configured with different protruding lengths from each other, so that the distal end of the plurality of coupling pins 423 form a concave shape as a whole to focally deliver ultrasound to an object.

As a third embodiment of the coupler module 400 (third coupler module 430), as shown in FIG. 10(b), the third coupler module 430 includes a planar coupler module main body 431 and a plurality of coupling pins 433 extending from the coupler module main body 431 toward the front surface, wherein the distal end of the plurality of coupling pins 433 form a concave shape, but protruding to different lengths from the plurality of coupling pins 423 according to the second embodiment. Therefore, the contact surface of the third coupler module 430 has a concave shape, different from the shape of the second coupler module 420. Thus, the third coupler module 430 may yield different focusing or dispersion direction of ultrasound compared to the second coupler module 420.

Like the first coupler module 410, the second coupler module 420, and the third coupler module 430, the focusing or dispersion direction of ultrasound propagation can be controlled by adjusting the curvature of the surface that contacts the scalp A or skin A, or by modifying the surface shape where each coupler module 400 is coupled to the transducer main body 100. Users can determine the desired focusing or dispersion direction of ultrasound by modifying the design of various embodiments of coupler modules 410, 420, 430 on the front surface of the transducer main body 100.

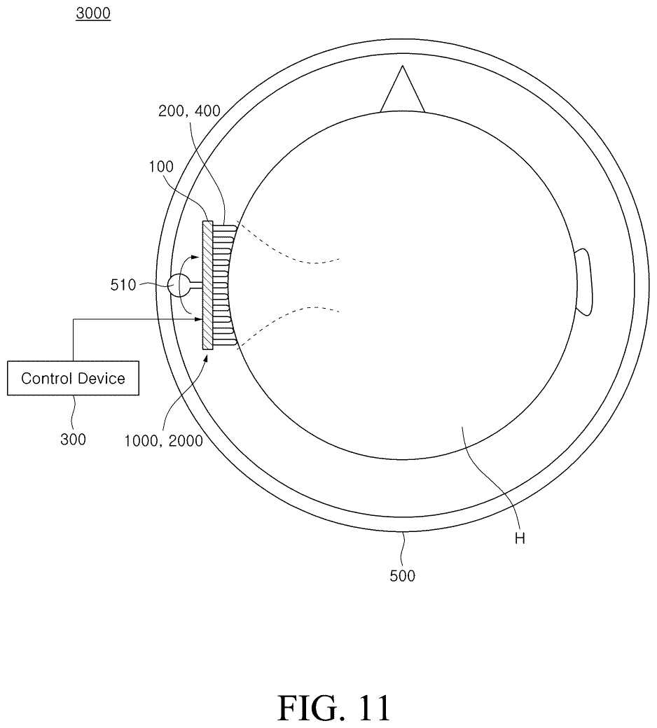

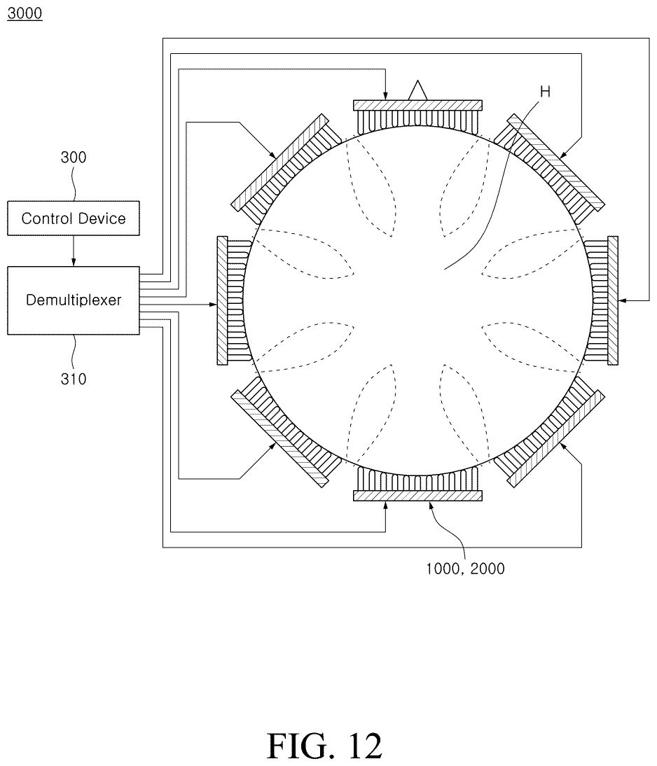

FIGS. 11 and 12 are views showing a wide-area transcranial ultrasound sonication device 3000 according to the present invention.

Referring to FIGS. 11 and 12, the wide-area transcranial ultrasound sonication device 3000 according to the present invention includes headgear 500 and ultrasonic transducers 1000, 2000 installed on the headgear 500.

The headgear 500 is worn on the head H. The ultrasonic transducers 1000, 2000 are installed on the headgear 500 and deliver ultrasound to biological tissue beneath the scalp H.

The ultrasonic transducers 1000, 2000 include the ultrasonic transducer 1000 described with reference to FIGS. 1, 3, and 6, and the ultrasonic transducer 2000 described with reference to FIGS. 7 to 10. For example, the ultrasonic transducers 1000, 2000 include a transducer main body 100 and a coupler 200, or a transducer main body 100 and a coupler module 400. The transducer main body 100, coupler 200, and coupler module 400 include the same configurations as described above, therefore, detailed descriptions are omitted.

The ultrasonic transducers 1000, 2000 are mounted inside of the headgear 500 by a rotatable swivel connector 510, enabling angle adjustment within the headgear 500 according to the shape of the skull H of the sonicated object.

The wide-area transcranial ultrasound sonication device 3000, according to the present invention, may further include an elastic body (not shown), such as a spring between the swivel connector 510 and the headgear 500, to gently apply pressure to the ultrasonic transducers 1000, 2000, allowing for making a closer contact with the scalp H. The control device 300 is connected to the ultrasonic transducers 1000, 2000. The control device 300 may further include an amplifier (not shown) that amplifies the electrical signal to actuate the ultrasonic transducers. For reference, although the control device 300 is shown as being installed outside headgear 500 in FIGS. 11 and 12, it may also be directly incorporated into the headgear 500, along with the ultrasonic transducers 1000, 2000.

As shown in FIG. 12, the wide-area transcranial ultrasound sonication device 3000 according to the present invention may further include a plurality of ultrasonic transducers 1000, 2000 installed within the headgear 500. For reference, in FIG. 12, the illustration of the headgear 500 is omitted.

As a plurality of ultrasonic transducers 1000, 2000 are installed within the headgear 500, ultrasound can be delivered to regions across the entire brain. The plurality of ultrasonic transducers 1000, 2000 may be connected to a single control device 300 and a 1-to-n signal demultiplexer 310.

Specifically, to deliver ultrasound to extensive brain areas, a plurality of transducers 1000, 2000 are installed within the headgear 500 to surround the brain, and the electrical signal from a single control device 300 and amplifier is split through a 1-to-n signal demultiplexer 310 circuit (with n equal to the number of transducers) and routed to each transducer 1000, 2000 to actuate it 1000, 2000. This configuration enables time-multiplexed pulsed operation of each transducer 1000, 2000, and minimizes power consumption by using a single amplifier. FIG. 12 illustrates an example in which eight transducers 1000, 2000 surround the brain. When a demultiplexer 310 with a 2 Hz switching frequency is used, each transducer channel can operate with a pulse duration (PD) of 62.5 ms.

Although ultrasound may be delivered continuously without stopping (duty cycle 100%), for transcranial Doppler imaging, functional neuromodulation, and transient blood-brain barrier opening, ultrasound is typically applied in a pulsed manner. To achieve pulsing of ultrasound, each transducer 1000, 2000 operates periodically with a pulse duration (PD) and pulse repetition frequency (PRF), and the duty cycle is determined as a percentage of PD (millisecond)×PRF (Hz)/1000 (millisecond).

In addition, the transducers 1000, 2000 need not be circular disks, and may be arranged to surround the head by combining geodesic forms of hexagons and pentagons.

According to an embodiment of the present invention, by employing a dry coupling method that does not require water-based coupling materials, (1) transcranial ultrasound delivery can be achieved while minimizing distortion in ultrasound propagation in the medium by spacing a plurality of coupling pins distributed at intervals of less than ½ of the ultrasound wavelength λ, (2) skin irritation can be avoided by using compressible flexible pin material and distributing pressure across a plurality of coupling pins, thereby enabling comfortable transcranial application of ultrasound for extended periods.

We note that, since the scalp contact area is smaller than in hydrogel-based coupling, the dry-coupling technique will transmit acoustic power at reduced level, approximately 30-40%, depending on the area where the coupling pins contact the scalp.

The dry acoustic coupling method and device of the present invention enable ultrasound delivery across a wide range of brain regions, including focal brain areas, by connecting the electrical signal output from a single amplifier to a plurality of transducers through a signal demultiplexer. This demultiplexed drive scheme, which connects multiple transducers to the output of a single amplifier, eliminates the need for separate amplifiers for each transducer, thereby reducing power consumption and enabling miniaturization of the ultrasound generator.

The scope of protection of the present invention is not limited to the description and expression of the embodiments explicitly described above. Furthermore, the scope of protection of the present invention may not be limited by obvious changes or substitutions in the technical field to which the present invention belongs.

What is claimed is:

1. An ultrasonic transducer comprising:
a main body that generates ultrasound; and
a coupler that transmits the ultrasound generated by the main body to a sonication target,
wherein the coupler includes a plurality of coupling pins protruding from a front surface of the main body to directly contact the sonication target.

2. The ultrasonic transducer of claim 1, wherein each of the plurality of coupling pins is made of a flexible material to be deformable when contacting the sonication target.

3. The ultrasonic transducer of claim 1, wherein a distal end portion of each of the plurality of coupling pins is formed with a curved surface having a predetermined radius-of-curvature.

4. The ultrasonic transducer of claim 1, wherein the plurality of coupling pins are arranged spaced apart from each other at spatially equal intervals.

5. The ultrasonic transducer of claim 1, wherein each of the plurality of coupling pins is spaced at intervals between 1/10 and ½ of the ultrasound wavelength in the sonication target.

6. The ultrasonic transducer of claim 1, wherein:
the main body includes a planar piezoelectric element,
the plurality of coupling pins are configured with different protruding lengths from each other, and
distal end portions of the plurality of coupling pins form a concave shape to focus the ultrasound.

7. The ultrasonic transducer of claim 1, wherein:
the main body includes a focused piezoelectric element that is concave toward the sonication target to focus the ultrasound, and
distal end portions of each of the plurality of coupling pins share the same plane.

8. An ultrasonic transducer comprising:
a main body that generates ultrasound; and
a dry acoustic coupler module mounted to a front surface of the main body,
wherein the coupler module includes:
a main body detachably mounted to the front surface of the transducer; and
a plurality of coupling pins protruding from the module for direct contact with scalp or skin.

9. The ultrasonic transducer of claim 8, wherein the coupler module includes a first coupler module in which contact surfaces of the plurality of coupling pins share the same plane.

10. The ultrasonic transducer of claim 8, wherein the coupler module includes a second coupler module in which end portions of the plurality of coupling pins form a concave shape.

11. The ultrasonic transducer of claim 10, wherein:
the coupler module further includes a third coupler module in which distal end portions of the plurality of coupling pins form a concave shape, and
the plurality of coupling pins of the third coupler module protruding to different lengths compared to those of the second coupler module and have a different concave shape of the second coupler module.

12. A wide-area transcranial ultrasound sonication device comprising:
headgear; and
an ultrasonic transducer installed on the headgear and delivering ultrasound to biological tissue beneath the scalp,
wherein the ultrasonic transducer includes:
a main body having a piezoelectric element that receives electrical signals to generate the ultrasound; and
a dry acoustic coupler attached to a front surface of the main transducer body and transmitting ultrasound to the biological tissue,
wherein the dry acoustic coupler includes a plurality of coupling pins spatially arranged to contact the scalp directly.

13. The wide-area transcranial ultrasound sonication device of claim 12, wherein the ultrasonic transducer is connected to the headgear by a swivel connector on an inside of the headgear, enabling angle adjustment to conform to the curvature of the skull.

14. The wide-area transcranial ultrasound sonication device of claim 12, wherein a plurality of ultrasonic transducers are mounted on the headgear.

15. The wide-area transcranial ultrasound sonication device of claim 14, wherein the plurality of ultrasonic transducers are connected to a single control device through a 1-to-n signal demultiplexer.
